(19) United States
(12) Patent Application Publication
(10) Pub. No.: US 2019/0142343 A1
(43) Pub. Date: May 16, 2019

(54) Reducing False Alarms in Patient Monitoring

(71) Applicant: Welch Allyn, Inc., Skaneateles Falls, NY (US)

(72) Inventors: Kirsten Emmons, Batesville, IN (US); Yongji Fu, Harrison, OH (US); Eric P. Jensen, Auburn, NY (US); Brian Lawrence, Cincinnati, OH (US); David Ribble, Indianapolis, IN (US); Duane Wiedor, Skaneateles Falls, NY (US)

(21) Appl. No.: 16/184,364

(22) Filed: Nov. 8, 2018

Related U.S. Application Data
(60) Provisional application No. 62/584,328, filed on Nov. 10, 2017.

(51) Int. Cl. A61B 5/00; A61B 5/0205; A61B 5/0452; A61B 5/04

(52) U.S. Cl. CPC A61B 5/721; A61B 5/746; A61B 5/0205; A61B 5/024; A61B 5/0452; A61B 5/04018; A61B 5/6892

(57) ABSTRACT

A system for providing continuous monitoring of a patient while reducing false alarms includes a first monitor device configured to measure one or more physiological attributes associated with the patient, a second monitor device configured to measure patient motion, and a processor coupled with the first monitor device and the second monitor device. The processor is configured to generate an alarm, based on data from the first monitor device corresponding to the one or more physiological attributes, and is further configured to suppress the alarm, based at least in part on the patient motion measured by the second monitor device, to reduce false alarms.

### BACKGROUND

The continuous monitoring of patients can be labor-intensive and time consuming. This is particularly true of patients who are recovering from surgery. The costs associated with caregivers monitoring the vital signs of post-surgical patients can be significant. This limits the number of patients that can be monitored by each caregiver and increases the medical costs. Automated systems used to provide such monitoring are sometimes inaccurate. Such systems can be plagued with inadequate monitoring and false alarms.

In many clinical environments, such as hospital rooms, skilled nursing facilities and the like, an overabundance of alarms on patient monitors is prevalent. Over-alarming is not only a nuisance for caregivers and patients, but it can also significantly and negatively impact patient care. So called “nuisance alarms” are those which do not require a clinical intervention. By some measures, as much as 95 percent of alarms in some clinical environments are nuisance alarms.

Therefore, reducing the prevalence of false and nuisance alarms would be advantageous for patient care in many different settings.

### SUMMARY

This application describes systems and methods for reducing false alarms in patient monitoring and/or improving accuracy of patient monitoring. In many embodiments described herein, patient motion is detected and used to filter patient monitoring data, and this filtering leads to more accurate patient information and a reduction in false alarms. In various other embodiments, other criteria or other forms of acquired patient data (sensed data or otherwise) may be used in addition to, or as an alternative to, patient motion to reduce false alarms.

Examples of different types of patient monitoring that often produces false alarms include electrocardiogram (ECG) monitoring and/or blood oxygen saturation/pulse oximeter (SpO2) monitoring. Motion artifact can be one of the leading causes of false alarms in ECG monitoring and SpO2 monitoring. For example, a hospital patient who is in bed and being monitored with a device may generate frequent false alarms that appear to indicate the patient is having a cardiac arrhythmia or low oxygen saturation, when actually the patient simply moved in bed.

In some embodiments disclosed in this application, various systems and methods are used to filter patient motion data out of ECG data and/or SpO2 data to help reduce false alarms and thus improve accuracy of monitoring. Alternative embodiments may use patient movement to reduce false alarms in other types of physiological monitoring, such as respiratory rate.

In one aspect of the present disclosure, a system for providing continuous monitoring of a patient while reducing false alarms may include a first monitor device, a second monitor device and a processor. The first monitor device is configured to measure one or more physiological attributes associated with the patient. The second monitor device is configured to measure patient motion. The processor is coupled with the first monitor device and the second monitor device and is configured to generate an alarm, based on data from the first monitor device corresponding to the one or more physiological attributes, and to suppress the alarm, based at least in part on the patient motion measured by the second monitor device, to reduce false alarms.

In various embodiments, the first monitor device may be an electrocardiogram (ECG) device, a pulse oximetry device, a blood pressure measurement device, a respiratory rate sensor or any other suitable physiological measurement device. The second monitor device may be any suitable patient motion detection device, such as a motion sensor device incorporated into or positioned on/under a patient support system, such as bed or chair. In one embodiment, for example, the motion detection may be a bed motion sensor that uses piezoelectric sensors or load cells to sense patient movement. In some embodiments, the first monitor device is a contact monitor device (i.e., is in contact with the patient), and the second monitor device is a non-contact monitor device (i.e., is not in contact with the patient).

The processor may be positioned in any suitable location, such as on a network coupled with the first and second monitor devices or in a central station coupled with the first and second monitor devices via a network. In some embodiments, the processor suppresses the alarm by filtering false data from the first monitor device, based on the patient motion. Alternatively, the processor may suppress the alarm by suppressing an alarm condition identified by the first monitor device, based on the patient motion. In some embodiments, suppression of the alarm by the processor is limited to a specified time period.

In another aspect of the present disclosure, a system for providing ECG monitoring of a patient while reducing false alarms may include an ECG device, a patient motion sensor device configured to measure patient motion and a processor coupled with the ECG device and the patient motion sensor device. The processor is configured to generate an alarm, based on data from the ECG device corresponding to a heart arrhythmia of the patient, and to suppress the alarm, based at least in part on the patient motion measured by the patient motion sensor device, to reduce false alarms.

In another aspect of the disclosure, a method for providing ECG monitoring of a patient while reducing false alarms may involve: measuring an ECG of the patient using an ECG device; detecting an alarm condition by the ECG device; measuring motion of the patient, using a patient motion detection device; and suppressing the alarm condition, using a processor coupled with the ECG device and the motion detection device, based at least in part on the measured motion of the patient.

In some embodiments, suppressing the alarm condition involves filtering false arrhythmia data from the ECG device, based on the patient motion. In some embodiments, the patient motion detection device includes a piezoelectric device and/or a load cell device. In some embodiments, the piezoelectric device or a load cell device is positioned under a mattress of a bed upon which the patient is laid.

These and other aspects and embodiments are described in further detail below, in relation to the attached drawing figures.

### DESCRIPTION OF THE FIGURES

FIG. 1 is a diagram illustrating a patient monitoring system including patient motion detection to reduce false alarms, according to one embodiment;

FIG. 2 is a block diagram of the patient monitoring system of FIG. 1;

FIG. 3 is a flow diagram illustrating a method for continuous monitoring of a patient while reducing false alarms, according to one embodiment;

FIG. 4 is a flow diagram illustrating a method for continuous monitoring of a patient while reducing false alarms, according to another embodiment;

FIG. 5 is a chart with blood pressure data indicative of movement of the patient, according to one embodiment;

FIG. 6 is a diagram illustrating use of data from a motion detection device to filter data from and ECG device and a pulse oximetry device, according to one embodiment; and

FIG. 7 is a diagram illustrating use of data from a motion detection device to filter data from and heart rate monitor and a pulse oximetry device, according to one embodiment.

### DETAILED DESCRIPTION

The present disclosure relates to systems and methods for reducing false alarms in patient monitoring and/or for improving accuracy of patient monitoring by accounting for patient motion. Patient motion is one of the most common causes of inaccuracy in many different types of patient monitoring, such as vital signs monitoring, including pulse oximetry, ECG (including telemetry monitoring and Holter monitoring), and newer contactless monitors. Most currently available patient monitoring technologies do not have the ability to account for patient motion, which in some cases leads to inaccurate monitoring data. Although the following description focuses on using patient motion to filter patient monitoring data, in various embodiments other forms of acquired patient data may be used to filter data to prevent false alarms and/or improve patient monitoring accuracy.

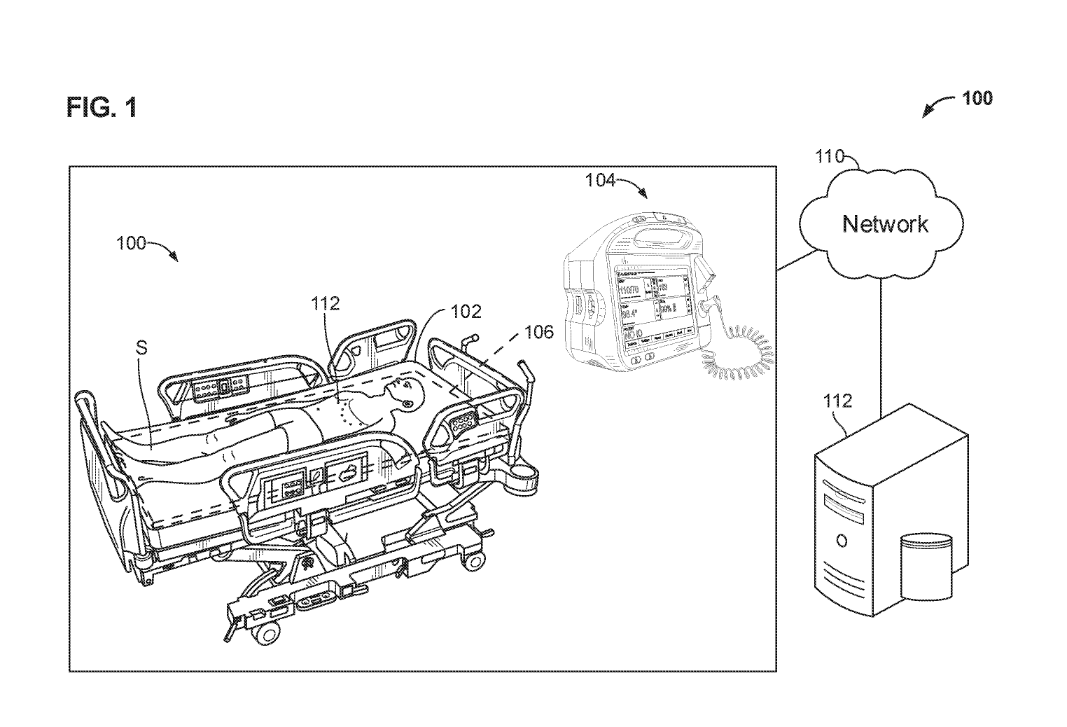

FIG. 1 schematically illustrates one embodiment of a patient monitoring system 100 for monitoring a subject S. In this example, the patient monitoring system 100 includes a motion detection device 106, a sensor device 112, and a physiological monitor device 104.

In one example, the sensor device 112 is an electrocardiogram (ECG) sensor device and/or a blood oxygen saturation/pulse oximeter (SpO2) sensor device. Various other types of sensor devices for measure other types of vitals can also be used. In the example of FIG. 1, the sensor device 112 is described as an ECG sensor device, although the disclosure is not so limited.

In the context of an ECG sensor device, the sensor device 112 typically includes multiple leads, which are placed on the patient's thorax to sense cardiac electrical signals. The sensor device 112 may communicate wirelessly or via wired connection with the monitor device 104. The monitor device 104 may, in some embodiments, also communicate with one or more other physiological sensing devices, such as a blood pressure monitor, a pulse oximetry device and/or a respiratory rate monitor.

In the embodiment shown, the motion detection device 106 is a below-mattress motion sensor, and the patient support system 102 is a hospital bed. In other embodiments, the motion detection device 106 may be located in any other suitable patient support system or device, including but not limited to other types of beds, lifts, chairs, stretchers, and surgical tables. In various embodiments, the motion detection device 106 may be a motion sensor system located on top of, within or under the mattress of the patient support system 102. In some embodiments, the motion detection device may include one or more piezoelectric sensors, load cells or combinations thereof. In alternative embodiments, the motion device 106 may be incorporated into the sensor device 112 (and/or into one or more other physiological sensing devices). In other words, in such embodiments, the physiological sensing function and the motion detection function are combined in one device. Multiple such devices may be used on a given patient in some embodiments. For example, in one embodiment a combined ECG/motion detection device and a combined pulse oximetry/motion detection device may be used on a patient at the same time.

The physiological monitor device 104 may be any suitable monitoring device, such as the multi-parameter device illustrated in FIG. 1. In other embodiments, the physiological monitor device 104 may be a single-parameter device, such as an ECG monitor.

The subject S can be a person, such as a patient, who is clinically treated by one or more healthcare practitioners. The healthcare practitioner is a person who provides healthcare service to the subject. Examples of healthcare practitioners include primary care providers (e.g., doctors, nurse practitioners, and physician assistants), nursing care providers (e.g., nurses), specialty care providers (e.g., professionals in various specialties), and health professionals that provide preventive, curative, promotional and rehabilitative health care services. The healthcare practitioner can be an institution, company, business, and/or entity. In other embodiments, the subject S can be an animal or other living organism that can be monitored with the system of the present disclosure. Although the system 100 is primarily described with respect to a single subject, it is understood that multiple subjects can be monitored with the system of the present disclosure, either individually or in group.

With continued reference to FIG. 1, in some examples, the subject monitoring system 100 is operable to communicate with a data management system 108 via a data communication network 110. The data management system 108 operates to manage the subject's personal and/or medical information, such as health conditions and other information. The data management system 108 can be operated by the healthcare practitioner and/or a healthcare service provider, such as a hospital or clinic.

Some embodiments of the data management system 108 are configured to communicate with the physiological monitor 104 and/or the motion detection device located in the patient support system 102. For example, the physiological monitor 104 and the data management system 108 may be connected via the network 110 to transmit various data therebetween. In other examples, the physiological monitor 104 is capable of directly communicating with the data management system 108 to transmit measurement data (and other data associated with the subject S). In some examples, the data management system 108 operates to provide information that can be used to assist the subject S, the subject's guardian and/or the healthcare practitioner to provide suitable healthcare to the subject S. Examples of the data management system 108 include Connex® data management systems, available from Welch Allyn Inc., Skaneateles Falls, N.Y.

The data communication network 110 communicates digital data between one or more computing devices, such as among the motion detection device in the patient support system 102, the physiological monitor 104 and/or the data management system 108. Examples of the network 110 include a local area network and a wide area network, such as the Internet. In some embodiments, the network 110 includes a wireless communication system, a wired communication system, or a combination of wireless and wired communication systems. A wired communication system can transmit data using electrical or optical signals in various possible embodiments. Wireless communication systems typically transmit signals via electromagnetic waves, such as in the form of optical signals or radio frequency (RF) signals. A wireless communication system typically includes an optical or RF transmitter for transmitting optical or RF signals, and an optical or RF receiver for receiving optical or RF signals. Examples of wireless communication systems include Wi-Fi communication devices (such as utilizing wireless routers or wireless access points), cellular communication devices (such as utilizing one or more cellular base stations), Bluetooth, ANT, ZigBee, medical body area networks, personal communications service (PCS), wireless medical telemetry service (WMTS), and other wireless communication devices and services.

In operation, the sensor device 112 senses electrical signals from the subject's heart and transmits the sensed signal data to the physiological monitor device 104. The motion detection device 106 in the patient support system 102 is used to sense patient motion (for example using piezoelectric or load cell sensors in the motion detection device 106). Sensed patient motion data is transmitted to the physiological monitor device 104, which processes the sensed data using a processor and an algorithm, to identify motion that may be associated with a cause of false alarms (e.g., motion artifact). The physiological monitor 104 then determines whether to sound or otherwise indicate an alarm, based on the sensed and processed motion data. For example, the system 100 may identify patient activity (e.g., significant activity in upper body) that would be likely to cause a motion artifact that would make a reading from the sensor device 112 inaccurate. Based on that probable cause of the inaccuracy, the system 100 would flag that reading as likely inaccurate and exclude it from communication, not generate an alert, exclude it from its risk scoring algorithm, and/or the like. In other examples, the system 100 may identify patient activity that would likely cause a motion artifact that would make the reading(s) of an SpO2 (blood oxygen saturation/pulse oximeter) monitor, a respiration monitor, a blood pressure monitor, or any combination of physiological monitors inaccurate. Again, the system 100 would flag that reading (or multiple readings) as likely inaccurate and exclude it from communication, not generate an alert, exclude it from its risk scoring algorithm, and/or the like.

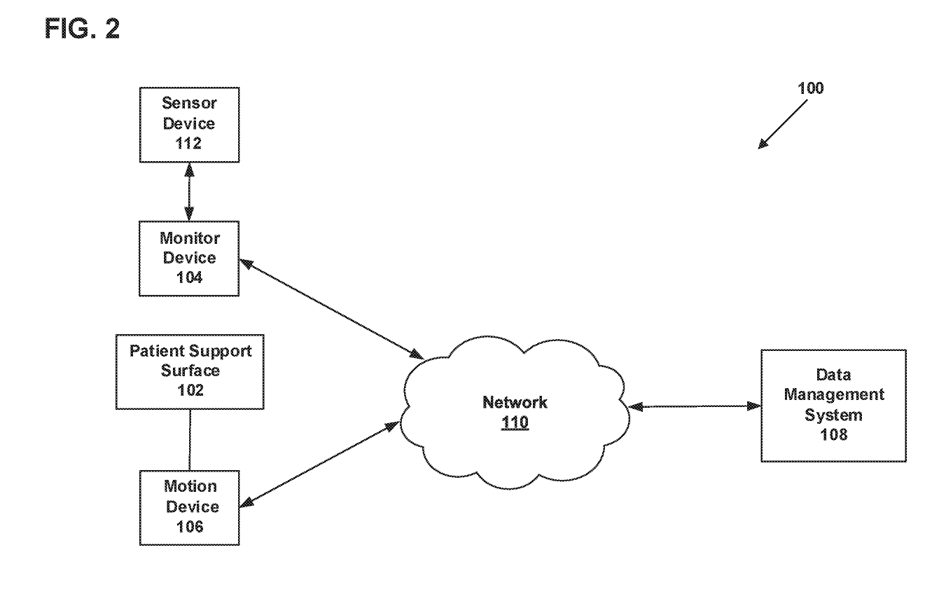

FIG. 2 is a block diagram illustrating the same patient monitoring system 100 as in FIG. 1. In this example, the patient is located in the patient support system 102. More broadly, the patient support system 102 may also be described as a “patient location,” which may be a hospital room or other location in which a patient is located for monitoring. In alternative embodiments, the patient support system 102 may be a chair, lift or any other suitable structure, as mentioned above. In other embodiments, the patient location may simply be a space in which the patient resides, such as a hospital room.

In this example, the patient is being monitored by two monitor devices—the sensor device 112 and the motion detection device 106, which may communicate via wired or wireless connection with the physiological monitor device 104. In some embodiments, for example where processing is performed outside of the monitor device 104, the motion device 106 may communicate directly with the network 110 rather than with the monitor device 104. In other embodiments, the motion device 106 may communicate with the network 110 and the monitor device 104. The sensor device 112 is used to monitor the patient's heartbeat and rhythm, and the motion detection device 106 is used to monitor patient motion. For example, the motion detection device 106 may be used to monitor whether the patient moves in bed or leaves the bed, or it may be used to monitor the patient's level of ambulation. In one embodiment, for example, the motion detection device 106 may be an in-bed motion sensor that resides above or below the mattress. Patient motion may be correlated with ECG monitoring, so that patient motion may be filtered out of the ECG data, to provide a more accurate measurement of heart rhythms.

In some embodiments, the patient may wear the sensor device 112 and the motion detection device 106. In such an embodiment, the level of ambulation can be measured as the patient sleeps, sits, stands, and moves. Some embodiments of such motion detection devices 106 may include one or more accelerometers. Other configurations are possible. As mentioned above, in other alternative embodiments, the sensor device 112 and the motion detection device 106 may be combined in one device, for example ECG electrodes with built-in accelerometer(s) or the like.

In alternative embodiments, the sensor device 112 may instead be a different type of physiological monitoring device. For example, the alternative device may be a contact-free patient monitor that does not directly contact the patient to measure the physiological attributes. It may use piezoelectric technology to monitor such attributes as heart rate, respiration rate, blood pressure, blood oxygen saturation and/or the like. In other embodiments, the monitoring device may contact the patient and monitor one or multiple attributes associated with a patient, such as temperature, blood oxygen saturation level (SpO2), non-invasive blood pressure (NIBP), end tidal carbon dioxide (ETCO2), and/or respiration rate. For example, the NIBP sensor may be a pressure cuff that is positioned around the patient's arm to take measurements to estimate such attributes as the patient's blood pressure, movement, heart rate, etc. In another example, the monitor device may include a photoplethysmography sensor used to create a photoplethysmogram. Such a sensor can be used to create a high-accuracy and resolution heart rate measurements. Any other suitable sensor or combination of sensors can be used in various embodiments.

The physiological monitor device 104 may be any suitable single-parameter or multi-parameter physiological monitoring device. In one embodiment, for example, the monitor device 104 is configured to monitor multiple physiological parameters of a patient, including ECG. In one example, the monitor device may be a Welch Allyn 1500 Patient Monitor, manufactured by Welch Allyn of Skaneateles Falls, N.Y. In other embodiments, the monitor device 104 may monitor only one parameter. For example, the monitor device 104 may simply be an ECG device in one embodiment, with the electrodes of the ECG device being attached to the patient and the monitor portion of the device residing apart from the patient.

In the illustrated embodiment, the monitor device 104 and the motion detecting device 106 communicate with a network 110. In one example, the monitor device 104, the motion detecting device 106 and the network 110 are part of a CONNEX™ System, from Welch Allyn of Skaneateles Falls, N.Y., although other systems can be used in various embodiments. In such an example, the monitor devices communicate through known protocols, such as the Welch Allyn Communications Protocol (WACP). WACP uses a taxonomy as a mechanism to define information and messaging. Taxonomy can be defined as description, identification, and classification of a semantic model. Taxonomy as applied to a classification scheme may be extensible. Semantic, class-based modeling, using taxonomy, can minimize the complexity of data description management by limiting, categorizing, and logically grouping information management and operational functions into families that contain both static and dynamic elements.

In some embodiments, the motion detection device 106 includes one or more motion detecting sensors housed in or on a patient motion bed sensor device. For example, the sensor of the motion detection device 106 can be placed under or on top of a mattress of the patient's bed located at the patient support system 102. Alternatively, the same or a similar motion detection device 106 may be positioned on or under a cushion of the patient's chair. In some embodiments, the motion detection device 106 may include one or more piezoelectric sensors or load cell sensors, which may allow the motion detection device 106 to sense patient motion without directly contacting the patient. In one embodiment, for example, the motion detection device 106 may be part of the EarlySense System, manufactured by EarlySense of Waltham, Mass. Aspects of that system are described in U.S. Patent Application Pub. No. 2007/0118054, filed on Oct. 25, 2006, which is hereby incorporated by reference in its entirety. In alternative embodiments, other motion detection devices 106 can be used.

The network 110 is an electronic communication network that facilitates communication between the monitor device 104 and the motion detecting device 106. An electronic communication network is a set of computing devices and links between the computing devices. The computing devices in the network use the links to enable communication among the computing devices in the network. The network 110 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various embodiments, the network 110 includes various types of links. For example, the network 110 can include wired and/or wireless links. Furthermore, in various embodiments, the network 110 is implemented at various scales. For example, the network 110 can be implemented as one or more local area networks (LANs), metropolitan area networks, subnets, wide area networks (such as the Internet), or can be implemented at another scale.

The monitor device 104 and the motion detecting device 106 communicate through the network 110 with a data management system 108. The data management system 108 may be positioned at a location at which a caregiver (e.g., a nurse or doctor) can monitor multiple patients. For example, in one embodiment the monitor device 104 and the motion detection device 106 send patient data to the data management system 108, and the caregiver monitors the patient information at the data management system 108. In one embodiment, the data management system 108 is a Welch Allyn Acuity® Central Monitoring Station, manufactured by Welch Allyn. Alternatively, any other suitable configuration is possible.

The monitor device 104 also provides alarm information to the data management system 108. For example, if the sensor device 112 detects a lack of heart rate or an arrhythmia (irregular heart rhythm) for a specified period of time, the monitor device 104 can communicate an alarm condition to the data management system 108. This alerts a caregiver at the data management system 108 of a condition that may require attention by the caregiver. In some instances, however, the alarm information is indicative of a false positive. Patient movement (e.g., rolling over, etc.) can cause loss of the ECG signal or a false signal. This slow acquisition time and loss of signal can result in alarm conditions that are false positives. For example, the patient could roll over, and the sensor device 112 could lose the acquisition of the signal associated with the patient's heart rate. If the signal is not reacquired in a certain amount of time, the monitor device 104 may provide an alarm condition to the data management system 108. This false positive may require a caregiver to check on the patient, wasting resources. Similar false positives may occur when the system 100 includes a different monitor (or monitors) than sensor device 112, such as an SpO2 sensor, respiratory rate sensor NIBP sensor or the like.

To minimize false positive information from being provided to the caregiver, data from the motion detection device 106 is used to filter out inaccurate data acquired from the sensor device 112 or to block an alarm generated by the sensor device 112. In one embodiment, for example, patient motion data detected by the motion detection device 106 may be used to filter inaccurate ECG data out of a heart rhythm measurement algorithm used by the sensor device 112, to help prevent arrhythmia false alarms. In some embodiments, a series of checks may be performed between the ECG monitor device 104 and the motion detection device 106. If the sensor device 112 indicates an alarm condition, for example, information from the motion detection device 106 may be checked prior to providing the alarm condition to the caregiver.

The system may also include a processor, which may be located on the network 110 or at the data management system 108, for example. The processor may be configured to filter ECG data, based on patient motion data. Alternatively or additionally, the processor may be configured to stop or block an alarm signal received from the sensor device 112, based at least part on patient motion data received from the motion detection device 106.

Further, as previously noted, the sensor device 112 can be used to sense other vital signs, such as pulse oximetry. In such an example, the motion data is used to filter erroneous alarms associated with SpO2 monitoring.

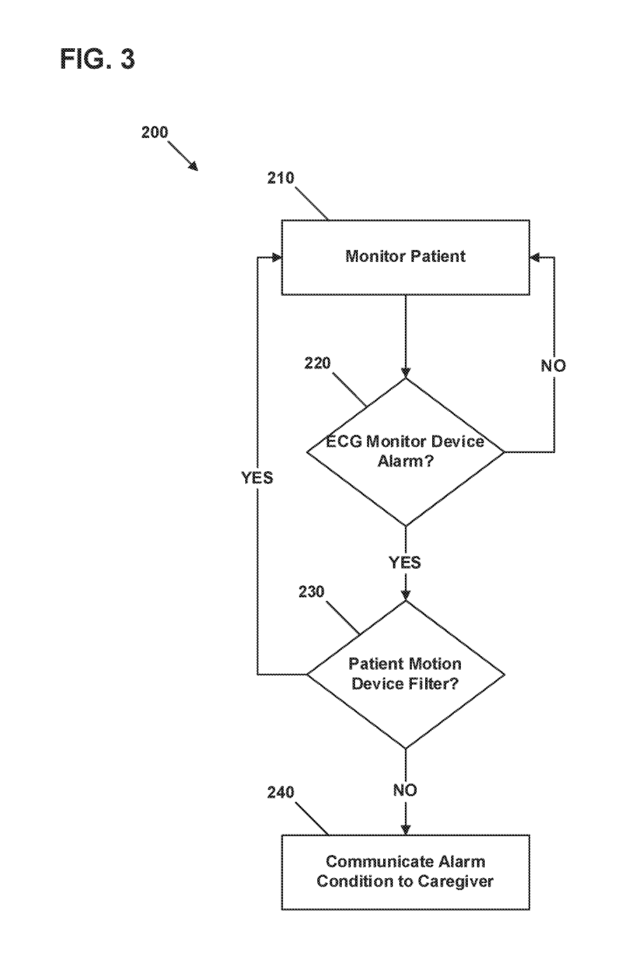

Referring now to FIG. 3, one embodiment of a method 200 for continuously monitoring a patient is provided while helping prevent false alarms is diagrammed. In this example, the patient is monitored at operation 210 using, for example, the ECG monitor device 104. Next, at operation 220, a determination is made regarding whether or not the sensor device 112 is providing an indication of an alarm condition. If not, control is passed back to operation 210, and monitoring is continued. If, on the other hand, the ECG monitor device 104 is indicating an alarm condition, control is instead passed to operation 230, and a determination is made regarding whether or not data from the motion detection device 106 will be used to filter out data from the sensor device 112 and thus negate the alarm. Or, alternatively, the data from the motion detection device 106 may be used to block the alarm. If so, control is passed back to operation 210, and continuous monitoring is continued. On the other hand, if there is no patient motion data or the system 100 determines that the patient motion data should not be used to filter or block the alarm from the sensor device 112, then control is passed to operation 240, and the alarm condition is communicated to the caregiver. For example, the alarm condition can be sent to the data management system 108.

Various aspects can be assessed when filtering at operation 230. For example, in one embodiment, the motion detection device sends one or more messages to the patient monitor according to the following example data schema.

Patient identifier

Motion level
Motion type

The patient identifier field can be a unique sequence of characters (e.g., 123456789) or other data that identifies the patient for the patient monitor. The motion level identifies or somehow quantifies the amount of motion for the patient. For example, when the motion sensor includes a load cell, the motion level can be represented on a scale of 0-10 as measured by the load cell. When the motion sensor is a blood pressure cuff (see below), the motion level can be a pressure level or change in level measured as measured in millimeters of mercury by the blood pressure cuff.

The motion type can be an optional field that attempts to quantify the type of motion measured by the motion device. In some examples, this can simply be another numerical representation using a range (e.g., 0 is sedentary; 1 is stirring; 2 is active motion). In another embodiment, the motion device can be programmed to provide a more specific representation of the type of motion (e.g., lying, sitting, walking, running). Other configurations are possible. The filtering can be performed based upon various algorithms.

The method 200 allows the motion detection device 106 to act as a check on the sensor device 112, to minimize false alarming. For example, if the sensor device 112 loses the heart rate due to patient movement and starts to alarm, the motion detection device 106 is consulted before the alarm condition is communicated to the caregiver at the data management system 108. In such a scenario, the method 200 uses the following logic.

| sensor device 112 alarm? | Motion detection device 106 filter? | Alarm? |
|---|---|---|
| Yes | No | Yes |
| No | Yes | No |
| Yes | Yes | No |
| No | No | No |

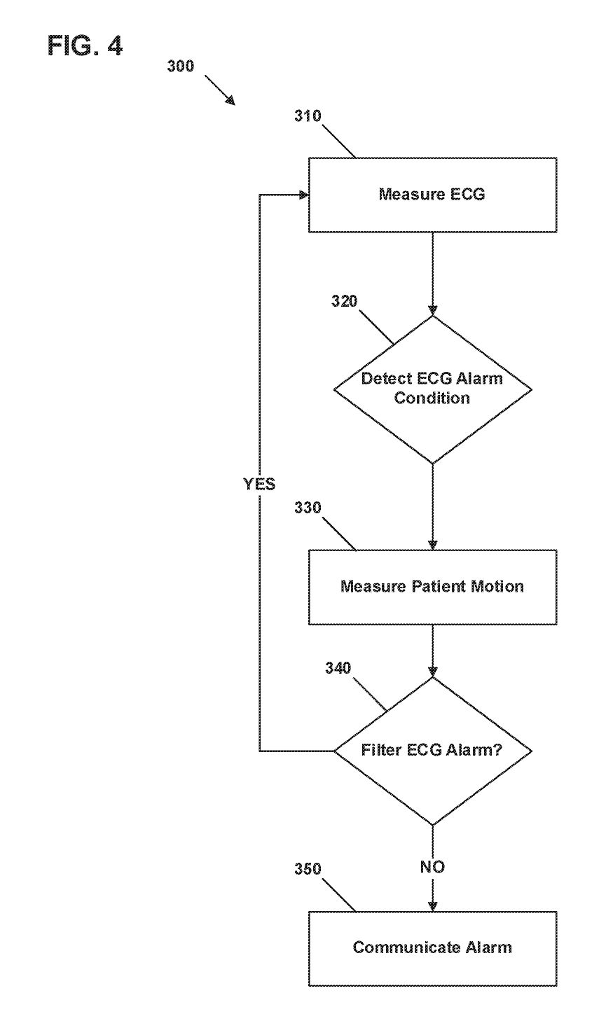

Referring now to FIG. 4, a method 300 for confirming an alarm condition during continuous monitoring of a patient is shown. At operation 310, the sensor device 112 measures cardiac rhythms of the patient. At operation 320, the sensor device 112 detects an ECG alarm condition. At operation 330 the motion detection device 106 measures patient motion. At operation 340, a determination is made regarding whether or not the measured patient motion data should be used to filter the ECG alarm condition—or in other words, whether an actual alarm condition exists. If the ECG alarm is not filtered (or blocked, or the like), then control is passed to operation 350, and the alarm is communicated. If the ECG alarm is filtered, control is passed back to operation 310, and the false positive is suppressed before the condition is used to alert the caregiver.

The alarming threshold for the system 100 can be configurable. For example, the sensitivity of the system 100 can be configured based upon different parameters, such as being dependent on the medication and/or surgical situation for the patient. For example, the system 100 can be set to be more sensitive, which may result in greater false positives but a closer level of supervision. The converse is true if the sensitivity is decreased.

For example, in some embodiments, the motion detection device 106 may include a non-invasive blood pressure (NIBP) measurement cuff, which may be inflated to a sub-measurement pressure (e.g., 30 mmHg through 300 mmHg). At this pressure, the NIBP cuff can register patient movement in the form of physical movement, breathing, and/or heart rate. This information can be used to make a determination of whether or not alarming is appropriate. For example, if the NIBP cuff is expanded and movement is detected, the alarm condition for the motion detection device 106 may be set to non-alarm, and the alarm condition from the sensor device 112 can be suppressed. Conversely, if no movement is detected, the alarm condition can be communicated to the caregiver.

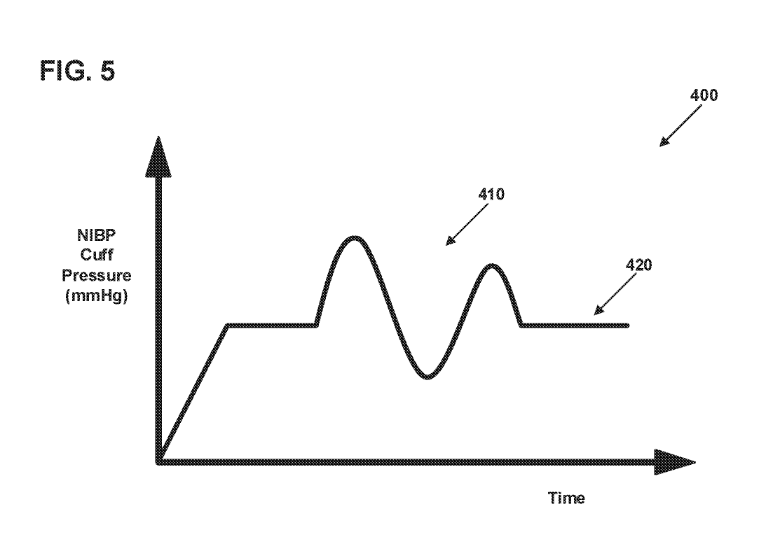

For example, FIG. 5 illustrates a chart 400 with data from a NIBP cuff that is plotted over time. The movement of the patient is manifested by the peaks and valleys shown at section 410. If the data is relative flat, such as at 420, the data would instead be indicative of no movement.

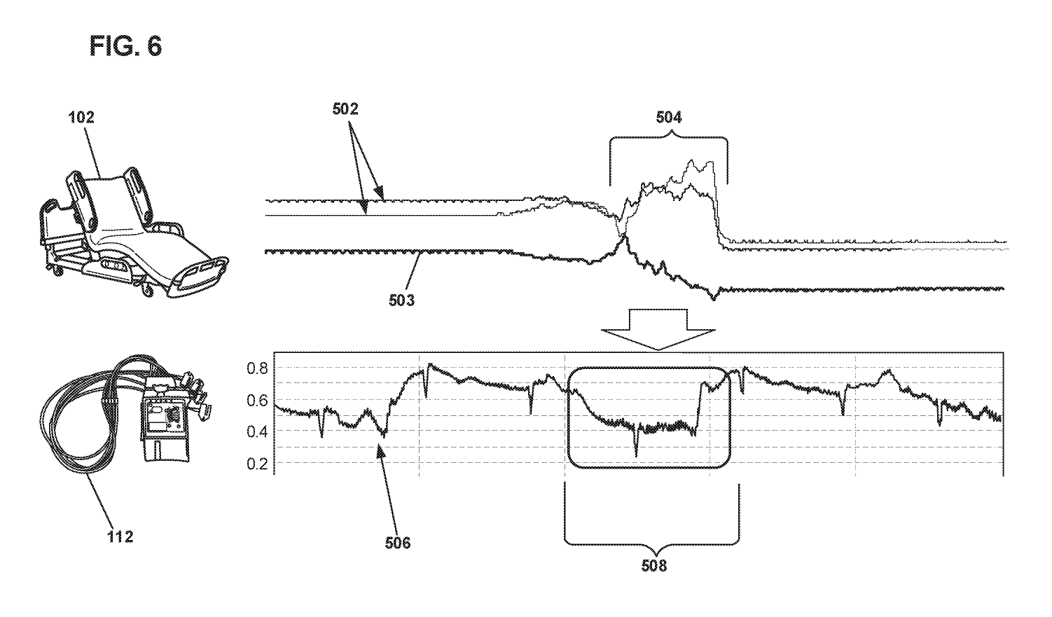

FIG. 6 illustrates one example, showing the patient support system 102 and sensor device 112 (telemetry monitor). In this example, the patient support system 102 includes a motion detection device 106, which is hidden by the mattress of the hospital bed. The patient is also being monitored with a pulse oximetry (SpO2) monitor in this example. As illustrated in the tracings on the right hand side of FIG. 6, a period of detected patient motion 504 (detected by the motion detection device 106), caused an increased heart rate in this patient, as shown by the two top heart rate tracings 502. Immediately below the heart rate tracings 502 is an SpO2 tracing 503, which shows that the SpO2 monitor detected that the patient's SpO2 decreased during the period of patient motion 504. The bottom tracing is an ECG tracing 506, which shows an abnormal heart rhythm period 508 during the period of patient motion 504. Using the system 100 and methods described herein, the detected patient motion may be used to filter the SpO2 decrease data and the ECG abnormal rhythm data, to prevent false alarms.

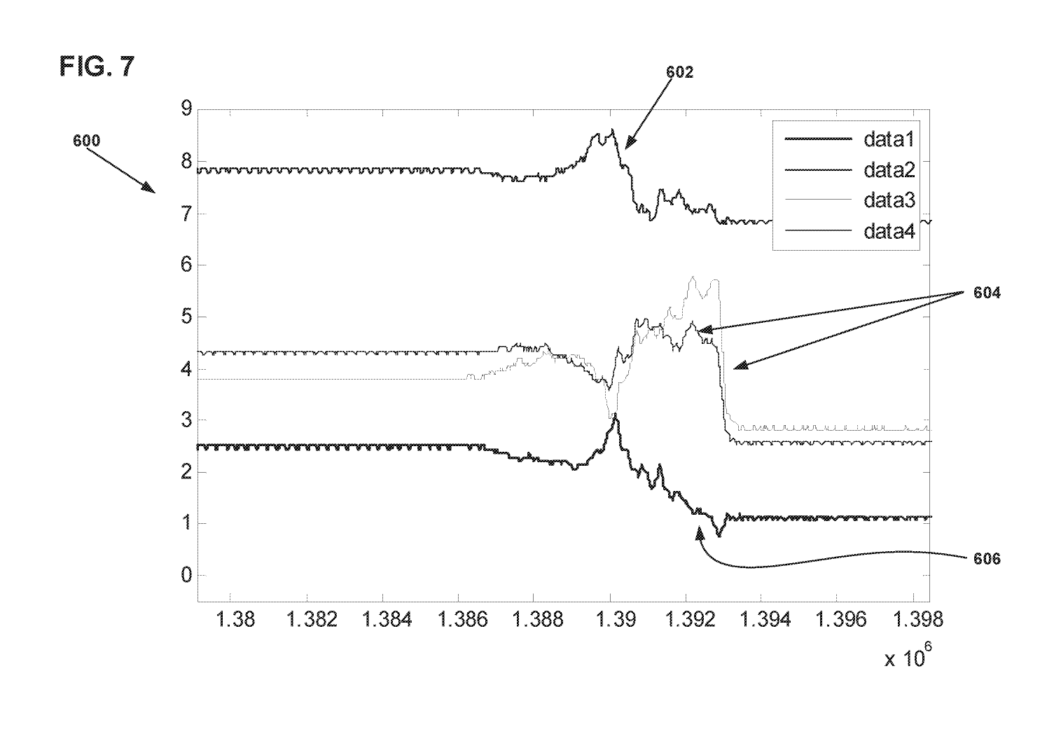

Referring to FIG. 7, another chart 600 is illustrated, showing similar results. Here, the top tracing is a patient motion tracing 602. An increase in patient motion (represented by the spike in the patient motion tracing 602) has caused a spike in the patient's heart rate to 110 beats per minute, illustrated as the two heart rate tracings 604, and a decrease in the patient's SpO2 to 90%, illustrated by the SpO2 tracing 606. Without the benefit of the technology described herein, the SpO2 system and the telemetry system would generate alerts, based on these vital signs being above the pre-defined thresholds. Using the system 100 and methods described herein, however, the system 100 would recognize that the SpO2 measurement 606 and heart rate measurements 604 were captured at a time when there was significant patient motion 602 and that the readings likely resulted from motion artifact. The system 100 would flag those measurements, and the measurements would be deemed inaccurate and excluded, to prevent false alarms.

In another example, the motion detection device 106 itself may include an SpO2 sensor, which may be used to determine whether an alarm condition exists. In this example, the SpO2 data is examined to look for indications of movement (e.g., spiking of the data) and/or heart rate (periodic). This information can be used to determine whether to suppress or allow the alarm condition to be communicated to the caregiver.

In some examples, the suppression of the alarm condition is time-based. For example, in one embodiment, if the sensor device 112 alarms and the motion detection device 106 senses movement, the motion detection device 106 only suppresses the alarm transmission to the caregiver for a certain period of time. If that period of time expires and the sensor device 112 continues to alarm, the alarm condition is transmitted to the caregiver even if the motion detection device 106 continues to sense an attribute that would allow for suppression of the alarm condition (e.g., patient movement).

In some examples, other factors can also be determined other than alarming conditions. For example, in another embodiment, the data from the devices is windowed or trended to determine a state of recovery for the patient—e.g., if a patient is starting to ambulate or worsen. These trends can be used to estimate a patient's progress, determine necessary interventions, and determine a proper discharge date.

The monitor device 104, the motion detection device 106 and the data management system 108 are computing devices. A computing device is a physical, tangible device that processes data. Example types of computing devices include personal computers, standalone server computers, blade server computers, mainframe computers, handheld computers, smart phones, special purpose computing devices, and other types of devices that process data.

Computing devices can include at least one central processing unit (“CPU”), a system memory, and a system bus that couples the system memory to the CPU. The system memory includes a random access memory (“RAM”) and a read-only memory (“ROM”). A basic input/output system containing the basic routines that help to transfer information between elements within the device, such as during startup, is stored in the ROM. The device further includes a mass storage device. The mass storage device is able to store software instructions and data.

The mass storage device and its associated computer-readable data storage media provide non-volatile, non-transitory storage for the device. Although the description of computer-readable data storage media contained herein refers to a mass storage device, such as a hard disk or CD-ROM drive, it should be appreciated by those skilled in the art that computer-readable data storage media can be any available non-transitory, physical device or article of manufacture from which the device can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the device.

The computing device can also include an input/output controller for receiving and processing input from a number of other devices, including a keyboard, a mouse, a touch user interface display screen, or another type of input device. Similarly, the input/output controller provides output to a touch user interface display screen, a printer, or other type of output device.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A system for providing continuous monitoring of a patient while reducing false alarms, the system comprising:
a first monitor device configured to measure one or more physiological attributes associated with the patient;
a second monitor device configured to measure patient motion; and
a processor coupled with the first monitor device and the second monitor device, wherein the processor is configured to generate an alarm, based on data from the first monitor device corresponding to the one or more physiological attributes, and is further configured to suppress the alarm, based at least in part on the patient motion measured by the second monitor device, to reduce false alarms.

2. The system of claim 1, wherein the first monitor device comprises a multi-parameter physiological monitor device, configured to monitor multiple physiological attributes of the patient.

3. The system of claim 1, wherein the first monitor device comprises an electrocardiogram (ECG) device.

4. The system of claim 1, wherein the one or more physiological attributes are selected from the group consisting of a respiratory rate, a heart rate, a heart rhythm, a blood oxygen saturation level and a blood pressure.

5. The system of claim 1, wherein the second monitor device comprises a bed motion sensor device coupled with a mattress of a patient bed in which the patient lies.

6. The system of claim 1, wherein the processor is located in at least one of a network coupled with the first and second monitor devices or a central station coupled with the first and second monitor devices.

7. The system of claim 1, wherein the processor suppresses the alarm by filtering false data from the first monitor device, based on the patient motion.

8. The system of claim 1, wherein the processor suppresses the alarm by suppressing an alarm condition identified by the first monitor device, based on the patient motion.

9. The system of claim 1, wherein suppression of the alarm by the processor is limited to a specified time period.

10. The system of claim 1, wherein the first monitor device comprises a contact monitor device, and the second monitor device comprises a non-contact monitor device.

11. The system of claim 10, wherein the non-contact monitor device comprises a piezoelectric device or load cell device positioned under a mattress of a bed upon which the patient is laid.

12. A system for providing electrocardiogram (ECG) monitoring of a patient while reducing false alarms, the system comprising:
an ECG device;
a patient motion sensor device configured to measure patient motion; and
a processor coupled with the ECG device and the patient motion sensor device, wherein the processor is configured to generate an alarm, based on data from the ECG device corresponding to a heart arrhythmia of the patient, and is further configured to suppress the alarm, based at least in part on the patient motion measured by the patient motion sensor device, to reduce false alarms.

13. The system of claim 12, wherein the patient motion sensor device comprises a bed motion sensor device coupled with a patient bed in which the patient lies.

14. The system of claim 12, wherein the processor suppresses the alarm by filtering false data from the ECG device, based on the patient motion.

15. The system of claim 12, wherein the processor suppresses the alarm by suppressing an alarm condition identified by the ECG device, based on the patient motion.

16. The system of claim 12, wherein suppression of the alarm by the processor is limited to a specified time period.

17. A method for providing electrocardiogram (ECG) monitoring of a patient while reducing false alarms, the method comprising:
measuring an ECG of the patient using an ECG device;
detecting an alarm condition by the ECG device;
measuring motion of the patient, using a patient motion detection device; and
suppressing the alarm condition, using a processor coupled with the ECG device and the motion detection device, based at least in part on the measured motion of the patient.

18. The method of claim 17, wherein suppressing the alarm condition comprises filtering false arrhythmia data from the ECG device, based on the patient motion.

19. The method of claim 17, wherein the patient motion detection device includes a piezoelectric device or load cell device.

20. The method of claim 19, wherein the piezoelectric device or load cell device is positioned under a mattress of a bed upon which the patient is laid.
